(19) United States
(12) Patent Application Publication
(10) Pub. No.: US 2009/0203140 A1
(43) Pub. Date: Aug. 13, 2009

(54) Genomic editing in zebrafish using zinc finger nucleases

(75) Inventors: SHARON AMACHER, BERKELEY, CA (US); YANNICK DOYON, EL CERRITO, CA (US); JASMINE MCCAMMON, BERKELEY, CA (US); FYODOR URNOV, POINT RICHMOND, CA (US)

Correspondence Address: ROBINS & PASTERNAK, 1731 EMBARCADERO ROAD, SUITE 230, PALO ALTO, CA, 94303, US

(73) Assignees: Sangamo BioSciences, Inc.; The Regents of the University of California

(21) Appl. No.: 12/284,897

(22) Filed: Sep. 25, 2008

Related U.S. Application Data
(60) Provisional application No. 60/995,577, filed on Sep. 27, 2007.
(60) Provisional application No. 61/068,207, filed on Mar. 5, 2008.
(60) Provisional application No. 61/125,817, filed on Apr. 29, 2008.

(51) Int. Cl. C12N 15/09; C12N 9/24; C12N 15/55; C12N 5/10

(52) U.S. Cl. 435/455; 435/200; 536/23.2; 435/325

(57) ABSTRACT

Disclosed herein are methods and compositions for genomic editing of one or more genes in zebrafish, using fusion proteins comprising a zinc finger protein and a cleavage domain or cleavage half-domain. Polynucleotides encoding said fusion proteins are also provided, as are cells comprising said polynucleotides and fusion proteins.

### CROSS-REFERENCE TO RELATED APPLICATIONS

The present application claims the benefit of U.S. Provisional Application No. 60/995,577, filed Sep. 27, 2007; U.S. Provisional Application No. 61/068,207, filed Mar. 5, 2008 and U.S. Provisional Application No. 61/125,817, filed September Apr. 29, 2008, the disclosures of which are hereby incorporated by reference in their entireties.

### STATEMENT OF RIGHTS TO INVENTIONS MADE UNDER FEDERALLY SPONSORED RESEARCH

Not applicable.

### TECHNICAL FIELD

The present disclosure is in the fields of genome engineering of zebrafish, including somatic and heritable gene disruptions, genomic alterations, generation of alleles carrying random mutations at specific positions of zebrafish genes and induction of homology-directed repair.

### BACKGROUND

Zebrafish (Danio rerio) have been widely used as model organisms as their embryonic development provides advantages over other vertebrate model organisms. Although the overall generation time of zebrafish is comparable to that of mice, zebrafish embryos develop rapidly, progressing from eggs to larvae in less than three days. The embryos are large, robust, and transparent and develop externally to the mother, characteristics which all facilitate experimental manipulation and observation. Their nearly constant size during early development facilitates simple staining techniques, and drugs may be administered by adding directly to the water. Mock fertilized eggs can be made to divide, and the two-cell embryo fused into a single cell, creating a fully homozygous embryo.

Currently, reverse genetics in zebrafish is generally accomplished using Morpholino antisense technology (commercially available from GeneTools). Morpholino oligonucleotides are stable, synthetic macromolecules that contain the same bases as DNA or RNA. When the antisense oligos bind to complementary RNA sequences they reduce the expression of specific genes. However, a known problem with genome editing in zebrafish is that, because the genome underwent duplication after the divergence of ray-finned fishes and lobe-finned fishes, it is not always easy to silence the activity of the two gene paralogs reliably with antisense oligos, due to complementation by the other paralog.

Thus, there remains a need for methods of modifying zebrafish genomes. Site-specific cleavage of genomic loci offers an efficient supplement and/or alternative to conventional homologous recombination. Creation of a double-strand break (DSB) increases the frequency of homologous recombination at the targeted locus more than 1000-fold. More simply, the imprecise repair of a site-specific DSB by non-homologous end joining (NHEJ) can also result in gene disruption. Creation of two such DSBs results in deletion of arbitrarily large regions. The modular DNA recognition preferences of zinc-fingers protein allows for the rational design of site-specific multi-finger DNA binding proteins. Fusion of the nuclease domain from the Type II restriction enzyme Fok I to site-specific zinc-finger proteins allows for the creation of site-specific nucleases. See, for example, United States Patent Publications 20030232410; 20050208489; 20050026157; 20050064474; 20060188987; 20060063231; and International Publication WO 07/014,275, the disclosures of which are incorporated by reference in their entireties for all purposes.

### SUMMARY

Disclosed herein are compositions for genomic editing in zebrafish, including, but not limited to: cleaving of one or more paralogs in zebrafish; targeted alteration (insertion, deletion and/or substitution mutations) in one or more zebrafish genes; the partial or complete inactivation of one or more paralogs in zebrafish; methods of inducing homology-directed repair and/or generation of random mutations encoding novel allelic forms of zebrafish genes.

In one aspect, described herein is a zinc finger protein (ZFP) that binds to target site in a region of interest in a zebrafish genome, wherein the ZFP comprises one or more engineered zinc finger binding domains. In one embodiment, the ZFP is a zinc finger nuclease (ZFN) that cleaves a target genomic region of interest in zebrafish, wherein the ZFN comprises one or more engineered zinc finger binding domains and a nuclease cleavage domain or cleavage half-domain. Cleavage domains and cleavage half domains can be obtained, for example, from various restriction endonucleases and/or homing endonucleases. In one embodiment, the cleavage half-domains are derived from a Type IIS restriction endonuclease (e.g., Fok I). The ZFN may specifically cleave one particular zebrafish gene sequence. Alternatively, the ZFN may cleave two or more homologous zebrafish gene sequences, which may include zebrafish paralogous or orthologous gene sequences.

The ZFN may bind to and/or cleave a zebrafish gene within the coding region of the gene or in a non-coding sequence within or adjacent to the gene, such as, for example, a leader sequence, trailer sequence or intron, or within a non-transcribed region, either upstream or downstream of the coding region. In certain embodiments, the ZFN binds to and/or cleaves a coding sequence or a regulatory sequence of the target zebrafish gene.

In another aspect, described herein are compositions comprising one or more of the zinc finger nucleases described herein. Zebrafish may contain one unique target gene or multiple paralogous target genes. Thus, compositions may comprise one or more ZFPs that target one or more genes in a zebrafish cell, for example, 1, 2, 3, 4, 5, or up to any number of paralogs or all paralogs present in a zebrafish cell. In one embodiment, the composition comprises one or more ZFPs that target all paralogous genes in a zebrafish cell. In another embodiment, the composition comprises one ZFP that specifically targets one particular zebrafish paralogous gene in a cell.

In another aspect, described herein is a polynucleotide encoding one or more ZFNs described herein. The polynucleotide may be, for example, mRNA.

In another aspect, described herein is a ZFN expression vector comprising a polynucleotide, encoding one or more ZFNs described herein, operably linked to a promoter.

In another aspect, described herein is a zebrafish host cell comprising one or more ZFN expression vectors. The zebrafish host cell may be stably transformed or transiently transfected or a combination thereof with one or more ZFP expression vectors. In one embodiment, the one or more ZFP expression vectors express one or more ZFNs in the zebrafish host cell.

In another aspect, described herein is a method for cleaving one or more paralogous genes in a zebrafish cell, the method comprising: (a) introducing, into the zebrafish cell, one or more polynucleotides encoding one or more ZFNs that bind to a target site in the one or more paralogous genes under conditions such that the ZFN(s) is (are) expressed and the one or more paralogous genes are cleaved. In one embodiment, one particular zebrafish paralogous gene in a zebrafish cell is cleaved. In another embodiment, more than one zebrafish paralog is cleaved, for example, 2, 3, 4, 5, or up to any number of paralogs or all paralogs present in a zebrafish cell are cleaved. The polynucleotide may be, for example, an mRNA.

In yet another aspect, described herein is a method for introducing an exogenous sequence into the genome of a zebrafish cell, the method comprising the steps of: (a) introducing, into the zebrafish cell, one or more polynucleotides encoding one or more ZFNs that bind to a target site in the one or more paralogous genes under conditions such that the ZFN(s) is (are) expressed and the one or more paralogous genes are cleaved; and (b) contacting the cell with an exogenous polynucleotide; such that cleavage of the paralogous genes stimulates integration of the exogenous polynucleotide into the genome by homologous recombination. In certain embodiments, the exogenous polynucleotide is integrated physically into the genome. In other embodiments, the exogenous polynucleotide is integrated into the genome by copying of the exogenous sequence into the host cell genome via nucleic acid replication processes (e.g., homology-directed repair of the double strand break). In certain embodiments, the one or more nucleases are fusions between the cleavage domain of a Type IIS restriction endonuclease and an engineered zinc finger binding domain.

In another embodiment, described herein is a method for modifying one or more gene sequence in the genome of a zebrafish cell, the method comprising (a) providing a zebrafish cell comprising one or more target gene sequences; and (b) expressing first and second zinc finger nucleases (ZFNs) in the cell, wherein the first ZFN cleaves at a first cleavage site and the second ZFN cleaves at a second cleavage site, wherein the gene sequence is located between the first cleavage site and the second cleavage site, wherein cleavage of the first and second cleavage sites results in modification of the gene sequence by non-homologous end joining. In certain embodiments, non-homologous end joining results in a deletion between the first and second cleavage sites. The size of the deletion in the gene sequence is determined by the distance between the first and second cleavage sites. Accordingly, deletions of any size, in any genomic region of interest, can be obtained. Deletions of 25, 50, 100, 200, 300, 400, 500, 600, 700, 800, 900, 1,000 nucleotide pairs, or any integral value of nucleotide pairs within this range, can be obtained. In addition deletions of a sequence of any integral value of nucleotide pairs greater than 1,000 nucleotide pairs can be obtained using the methods and compositions disclosed herein. In other embodiments, non-homologous end joining results in an insertion between the first and second cleavage sites. Methods of modifying the genome of a zebrafish as described herein can be used to create models of animal (e.g., human) disease, for example by inactivating (partially or fully) a gene or by creating random mutations at defined positions of genes that allows for the identification or selection of animals carrying novel allelic forms of those genes.

In yet another aspect, described herein is a method for germline disruption of one or more target genes in zebrafish, the method comprising modifying one or more gene sequences in the genome of one or more cells of a zebrafish embryo by any of the methods described herein and allowing the zebrafish embryo to reach sexual maturity, wherein that the modified gene sequences are present in at least a portion of gametes of the sexually mature zebrafish.

In another aspect, described herein is a method of creating one or more heritable mutant alleles in a zebrafish loci of interest, the method comprising modifying one or more loci in the genome of one or more cells of a zebrafish embryo by any of the methods described herein; raising the zebrafish embryo to sexual maturity; and allowing the sexually mature zebrafish to produce offspring; wherein some of the offspring comprise the mutant alleles.

### BRIEF DESCRIPTION OF THE DRAWINGS

FIG. 1 shows pigmentation of zebrafish embryos upon disruption of the golden gene. The top panel shows a wild-type organism. The second panel from the top shows a zebrafish embryo when the golden gene was mutated as described in Lamason et al. (2005) Science 310(5755):1782-6. The left most bottom panel shows eye pigmentation in zebrafish with a gol^{bl+/-} background. The 3 right bottom panels show eye pigmentation in gol^{bl+/-} zebrafish injected with 5 ng of ZFN mRNA directed against golden gene.

FIG. 2 is a graph depicting the percentage of zebrafish embryos displaying the indicated phenotype upon injection of ZFN mRNA of various golden-targeted ZFN pairs (indicating on the horizontal axis). The light gray bars show the percentage of wild-type eye pigmentation. The dark gray bars show the percentage of embryos having unpigmented eyes and the white bars show the percentage of embryos not scored.

FIG. 3 shows sequence analysis of cells from various zebrafish embryos injected with golden-targeted ZFN mRNAs. Deletions and insertions induced by the ZFNs are shown as indicated.

FIG. 4, panels A to D, show tail formation of zebrafish embryos upon disruption of the no tail/Brachyury (ntl) gene. FIG. 4A shows a wild-type zebrafish embryo. FIG. 4B shows a zebrafish embryo when the no tail gene was mutated as described in Amacher et al. (2002) Development 129(14):3311-23. FIG. 4C shows a zebrafish embryo with ntl^{+/-} genotype and FIG. 4D shows a zebrafish embryo with a ntl genotype injected with 5 ng of ZFN mRNA directed against the ntl gene.

FIG. 5, panels A to H, show tails of wild-type, ntl hypomorph and ntl-injected ZFN embryos. FIG. 5A shows an embryo injected with 5 ng ntl-targeted ZFN pairs. FIG. 5B shows ntl hypomorph ntl^{b487}. FIGS. 5C and D show a wild-type zebrafish embryo. FIGS. 5E and G show ntl hypomorphic phenotypes in ntl^{b195} heterozygous embyos following injection with 5 ng ntl encoding ZFN pairs. FIGS. 5F and H show ntl hypomorph ntl^{b487} embryos.

FIG. 6 shows sequence analysis of cells from various zebrafish embryos injected with ntl-targeted ZFNs. Deletions and insertions induced by the ZFNs are shown as indicated.

FIG. 7, panels A to C, show tail formation and partial sequence of no tail alleles in zebrafish injected with no tail-targeted ZFNs. FIG. 7A shows tail formation of wildtype uninjected zebrafish embryos (left panel) and zebrafish embryos injected with mRNA encoding ntl-targeted ZFNs (middle and left panels). Embryos showed ntl-like phenotypes (middle panel), and some showed additional mild necrosis (right panel). In situ hybridization of representative embryos to detect notochordal ntl expression are inset in each panel. FIG. 7B shows sequencing of the ntl locus of one representative ntl-targeting ZFN mRNA-injected embryo. As shown, a large number of unique ntl alleles were observed, and up to 70% of the sequenced chromatids carried an induced mutation. FIG. 7C shows sequencing of the ntl locus of small posterior tissue samples taken from tailless adult zebrafish (see, FIG. 8A) into which ntl-targeting ZFN mRNA was injected. The frequency of each allele type is indicated after the allele description.

FIG. 8, panels A to C, show tail formation and partial sequence data of ntl alleles of juvenile zebrafish derived from wildtype embryos injected with mRNA encoding ntl-targeting ZFNs with posterior truncations. FIG. 8A shows normal juveniles (two left-most panels) as well as posteriorly truncated juvenile zebrafish (two right most panels). FIG. 8B depicts ntl phenotypes observed in wild-type (left panel) zebrafish embryos and in progeny of ZFN-injected founder animals in complementation crosses (right panel). Wildtype embryos injected with mRNA encoding ntl-targeting ZFN were grown to adulthood and eggs from founder females were fertilized in vitro with sperm from ntl^{b195} heterozygous males for complementation crosses. FIG. 8C shows sequence data of ntl alleles from 4 founder animals that gave phenotypically ntl progeny in complementation cross.

### DETAILED DESCRIPTION

Described herein are compositions and methods for genomic editing in zebrafish (e.g., cleaving of genes; alteration of genes, for example by cleavage followed by insertion (physical insertion or insertion by replication via homology-directed repair) of an exogenous sequence and/or cleavage followed by non-homologous end joining (NHEJ); partial or complete inactivation of one or more genes; generation of alleles with random mutations to create altered expression of endogenous genes; etc.). Also disclosed are methods of making and using these compositions (reagents), for example to edit (alter) one or more genes in a target zebrafish cell. Thus, the methods and compositions described herein provide highly efficient methods for targeted gene alteration (e.g., knock-in) and/or knockout (partial or complete) of one or more zebrafish genes (paralogs) and/or for randomized mutation of the sequence of any target allele, and, therefore, allow for the generation of animal models of human diseases.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS 1N MOLECULAR BIOLOGY, Vol. 119, “Chromatin Protocols” (P. B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

A “selected” zinc finger protein is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage-half-domain” is a cleavage half-domain that has been modified so as to form obligate heterodimers with another cleavage half-domain (e.g., another engineered cleavage half-domain). See, also, U.S. patent application Ser. Nos. 10/912,932 and 11/304,981 and U.S. Provisional Application No. 60/808,486 (filed May 25, 2006), incorporated herein by reference in their entireties.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a cleavage domain, the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

Zinc Finger Nucleases

Described herein are zinc finger nucleases (ZFNs) that can be used for genomic editing (e.g., cleavage, alteration, inactivation and/or random mutation) of one or more zebrafish genes. ZFNs comprise a zinc finger protein (ZFP) and a nuclease (cleavage) domain (e.g., cleavage half-domain).

A. Zinc Finger Proteins

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

Selection of target sites; ZFPs and methods for design and construction of fusion proteins (and polynucleotides encoding same) are known to those of skill in the art and described in detail in U.S. Patent Application Publication Nos. 20050064474 and 20060188987, incorporated by reference in their entireties herein.

In addition, as disclosed in these and other references, zinc finger domains and/or multi-fingered zinc finger proteins may be linked together using any suitable linker sequences, including for example, linkers of 5 or more amino acids in length (e.g., TGEKP (SEQ ID NO:1), TGGQRP (SEQ ID NO:2), TGQKP (SEQ ID NO:3), and/or TGSQKP (SEQ ID NO:4)). See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Table 1 describes a number of zinc finger binding domains that have been engineered to bind to nucleotide sequences in a zebrafish golden gene and Table 4 shows the recognition helices of a number of zinc finger binding domains designed to bind to nucleotide sequences in a zebrafish no tail gene. In particular, the second through fourth columns show the amino acid sequence of the recognition region (amino acids −1 through +6, with respect to the start of the helix) of each of the zinc fingers (F1 through F4) in each protein. Each row describes a separate zinc finger DNA-binding domain. Also provided in the first column is an identification number for the proteins. The DNA target sequence for each protein is shown in Table 2 (golden designs) and Table 5 (no tail designs).

As described below, in certain embodiments, a four-, five-, or six-finger binding domain is fused to a cleavage half-domain, such as, for example, the cleavage domain of a Type IIs restriction endonuclease such as FokI. One or more pairs of such zinc finger/nuclease half-domain fusions are used for targeted cleavage, as disclosed, for example, in U.S. Patent Publication No. 20050064474.

For targeted cleavage, the near edges of the binding sites can separated by 5 or more nucleotide pairs, and each of the fusion proteins can bind to an opposite strand of the DNA target. All pairwise combinations 1 can be used for targeted cleavage of a zebrafish gene. Following the present disclosure, ZFNs can be targeted to any sequence in the zebrafish genome.

B. Cleavage Domains

The ZFNs also comprise a nuclease (cleavage domain, cleavage half-domain). The cleavage domain portion of the fusion proteins disclosed herein can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10, 570-10, 575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014,275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474 and 20060188987 and in U.S. application Ser. No. 11/805,850 (filed May 23, 2007), the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino-acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., Example 1 of U.S. Provisional Application No. 60/808,486 (filed May 25, 2006), the disclosure of which is incorporated by reference in its entirety for all purposes.

Engineered cleavage half-domains described herein can be prepared using any suitable method, for example, by site-directed mutagenesis of wild-type cleavage half-domains (Fok I) as described in U.S. Patent Publication No. 20050064474 (Ser. No. 10/912,932, Example 5) and U.S. Patent Provisional Application Ser. No. 60/721,054 (Example 38).

C. Additional Methods for Targeted Cleavage in Zebrafish

Any nuclease having a target site in a zebrafish gene can be used in the methods disclosed herein. For example, homing endonucleases and meganucleases have very long recognition sequences, some of which are likely to be present, on a statistical basis, once in a human-sized genome. Any such nuclease having a target site in a unique or paralogous zebrafish gene can be used instead of, or in addition to, a zinc finger nuclease, for targeted cleavage in a zebrafish gene or multiple paralogs.

Exemplary homing endonucleases include I-SceI, I-CeuI, PI-PspI, PI-Sce, I-SceIV, I-CsmI, I-PanI, I-Scell, I-PpoI, I-SceIII, I-CreI, I-TevI, I-TevIl and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

Although the cleavage specificity of most homing endonucleases is not absolute with respect to their recognition sites, the sites are of sufficient length that a single cleavage event per mammalian-sized genome can be obtained by expressing a homing endonuclease in a cell containing a single copy of its recognition site. It has also been reported that the specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66.

Delivery

The ZFNs described herein may be delivered to a target zebrafish cell by any suitable means, including, for example, by injection of ZFN mRNA. See, Hammerschmidt et al. (1999) Methods Cell Biol. 59:87-115

Methods of delivering proteins comprising zinc fingers are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

ZFNs as described herein may also be delivered using vectors containing sequences encoding one or more of the ZFNs. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more ZFN encoding sequences. Thus, when one or more pairs of ZFNs are introduced into the cell, the ZFNs may be carried on the same vector or on different vectors. When multiple vectors are used, each vector may comprise a sequence encoding one or multiple ZFNs.

Conventional viral and non-viral based gene transfer methods can be used to introduce nucleic acids encoding engineered ZFPs in zebrafish cells. Such methods can also be used to administer nucleic acids encoding ZFPs to zebrafish cells in vitro. In certain embodiments, nucleic acids encoding ZFPs are administered for in vivo or ex vivo uses.

Non-viral vector delivery systems include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids. Viral vector delivery systems include DNA and RNA viruses, which have either episomal or integrated genomes after delivery to the cell. Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

As noted above, the disclosed methods and compositions can be used in any type of zebrafish cell. Progeny, variants and derivatives of zebrafish cells can also be used.

### Applications

The disclosed methods and compositions can be used for genomic editing of any zebrafish gene or genes. In certain applications, the methods and compositions can be used for inactivation of zebrafish genomic sequences, for example paralogs of a zebrafish gene. In other applications, the methods and compositions allow for generation of random mutations, including generation of novel allelic forms of genes with different expression as compared to unedited genes, which in turn allows for the generation of animal models. In other applications, the methods and compositions can be used for creating random mutations at defined positions of genes that allows for the identification or selection of animals carrying novel allelic forms of those genes. In other applications, the methods and compositions allow for targeted integration of an exogenous (donor) sequence into any selected area of the zebrafish genome. By “integration” is meant both physical insertion (e.g., into the genome of a host cell) and, in addition, integration by copying of the donor sequence into the host cell genome via the nucleic acid replication processes. Genomic editing (e.g., inactivation, integration and/or targeted or random mutation) of a zebrafish gene can be achieved, for example, by a single cleavage event, by cleavage followed by non-homologous end joining, by cleavage followed by homology-directed repair mechanisms, by cleavage followed by physical integration of a donor sequence, by cleavage at two sites followed by joining so as to delete the sequence between the two cleavage sites, by targeted recombination of a missense or nonsense codon into the coding region, by targeted recombination of an irrelevant sequence (i.e., a “stuffer” sequence) into the gene or its regulatory region, so as to disrupt the gene or regulatory region, or by targeting recombination of a splice acceptor sequence into an intron to cause mis-splicing of the transcript. See, U.S. Patent Publication Nos. 20030232410; 20050208489; 20050026157; 20050064474; 20060188987; 20060063231; and International Publication WO 07/014,275, the disclosures of which are incorporated by reference in their entireties for all purposes.

There are a variety of applications for ZFN-mediated genomic editing of zebrafish. For example, the methods and compositions described herein allow for the generation of zebrafish models of human disease.

### EXAMPLES

### Example 1

### ZFNs Induce Targeted Disruption at the Golden/slc24a5 (gol) Locus

ZFNs targeted to various distinct positions in the golden/slc24a5 (gol), or hereafter, golden locus were designed and incorporated into plasmids essentially as described in Umov et al. (2005) Nature 435(7042):646-651. ZFN pairs were screened for activity in a yeast-based chromosomal system as described in U.S. Ser. No. 60/995,566, entitled “Rapid in vivo Identification of Biologically Active Nucleases.”

The recognition helices for representative golden zinc finger designs are shown below in Table 1.

TABLE 1
Golden Zinc finger Designs

| | | | | |
|---|---|---|---|---|
| ZFN Name | F1 | F2 | F3 | F4 |

| | | | | |
|---|---|---|---|---|
| 12775 golden | DRSDLSR | RSDDLTR | RSDDLTR | QSGDLTR |
| | (SEQ ID | (SEQ ID | (SEQ ID | (SEQ ID |
| | NO:5) | NO:6) | NO:6) | NO:7) |
| 12776 golden | TSGSLSR | RSDNLRE | RSDALSE | QNATRTK |
| | (SEQ ID | (SEQ ID | (SEQ ID | (SEQ ID |
| | NO:8) | NO:9) | NO:10) | NO:11) |
| 12804 golden | DRSHLSR | RSDALAR | DRSNLSR | TSGSLTR |
| | (SEQ ID | (SEQ ID | (SEQ ID | (SEQ ID |
| | NO:12) | NO:13) | NO:14) | NO:15) |
| 12805 golden | QSGNLAR | TSANLSR | RSDTLSE | RSQTRKT |
| | (SEQ ID | (SEQ ID | (SEQ ID | (SEQ ID |
| | NO:16) | NO:17) | NO:18) | NO:19) |
| 12806 golden | QSGNLAR | TSGNLTR | RSDTLSE | RSQTRKT |
| | (SEQ ID | (SEQ ID | (SEQ ID | (SEQ ID |
| | NO:20) | NO:21) | NO:18) | NO:19) |

Target sites of the golden zinc finger designs are shown below in Table 2.

TABLE 2
Target Sites of Golden Zinc Fingers

| | |
|---|---|
| ZFN Name | Target Site (5′ to 3′ ) |

| | |
|---|---|
| 12775 golden | gtGCAGCGtGCGGCCtgctgtcctctgc |
| | (SEQ ID NO:22) |
| 12776 golden | atGCACAGCAGGTTatagacagcagaac |
| | (SEQ ID NO:23) |
| 12804 golden | ttGTTGACGTGGGCtccccggtggatgt |
| | (SEQ ID NO:24) |
| 12805 golden | acCGTCTGGATGAAccacggcaggccca |
| | (SEQ ID NO:25) |
| 12806 golden | acCGTCTGGATGAAccacggcaggccca |
| | (SEQ ID NO:26) |

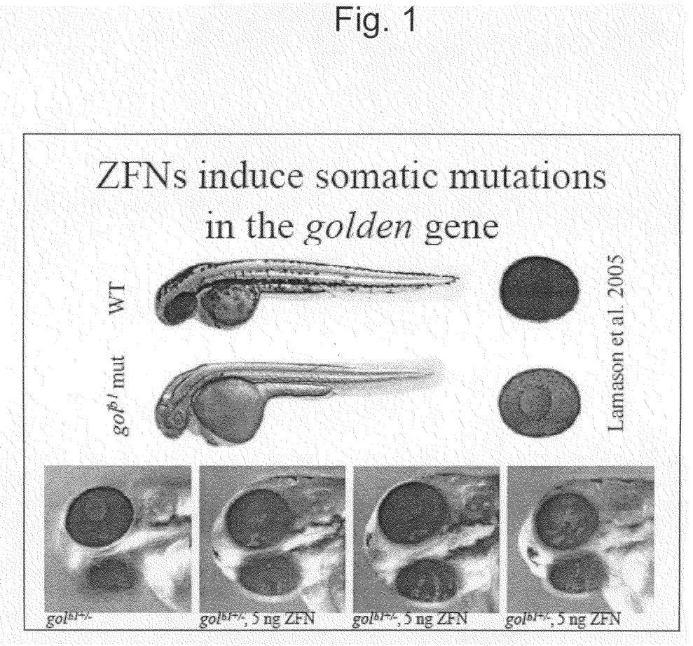

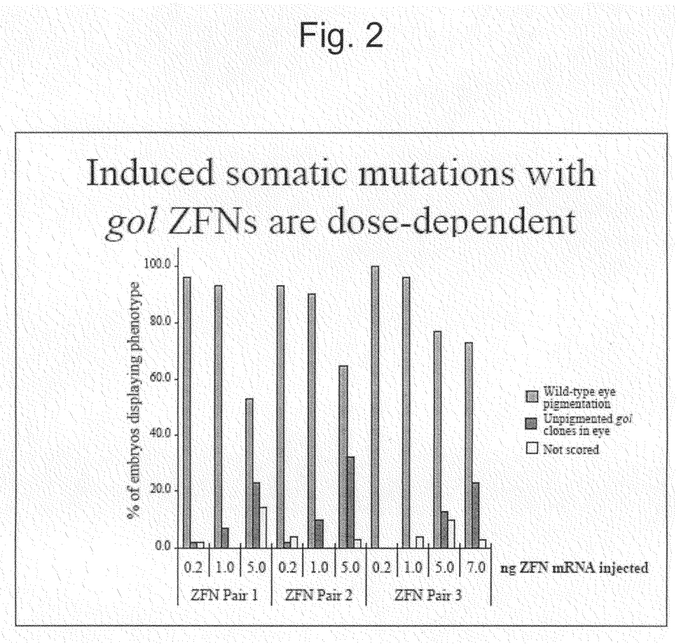

Active golden-targeted ZFN mRNA was introduced into zebrafish embryos by injection as described in Hammerschmidt et al. (1999) Methods Cell Biol. 59:87-115 and embryos evaluated for their pigmentation. Results are shown in Table 3 and FIGS. 1 and 2.

TABLE 3
ZFNs directed to the zebrafish golden gene induce somatic loss-of-function

| | | Wild-type eye pigmentation | | | Unpigmented gol clones in eye² | | | |
|---|---|---|---|---|---|---|---|---|
| ZFN | Dose | Normal | Abnormal | Total | Normal | Abnormal | | |
| pair¹ | (ng) | embryos | embryos³ | percentage | embryos | embryos³ | Total % | unscored⁴ |
| 04.05 | 0.2 | 54/58 | 2/58 | 96% | 1/58 | 0/58 | 2% | 1/58 |
| | | (93%) | (3%) | | (2%) | (0%) | | (2%) |
| | 1.0 | 39/42 | 0/42 | 93% | 2/42 | 1/42 | 7% | 0/42 |
| | | (93%) | (0%) | | (5%) | (2%) | | (0%) |
| | 5.0 | 26/49 | 0/49 | 53% | 11/49 | 5/49 | 32% | 7/49 |
| | | (53%) | (0%) | | (22%) | (10%) | | (14%) |
| 04.06 | 0.2 | 42/45 | 0/45 | 93% | 1/45 | 0/45 | 2% | 2/45 |
| | | (93%) | (0%) | | (2%) | (0%) | | (4%) |
| | 1.0 | 26/29 | 0 | 90% | 3/29 | 0 | 10% | 0/29 |
| | | (90%) | (0%) | | (10%) | (0%) | | (0%) |
| | 5.0 | 24/37 | 0 | 65% | 9/37 | 3/37 | 32% | 1/37 |
| | | (65%) | (0%) | | (24%) | (8%) | | (3%) |
| 75.76 | 0.2 | 11/11 | 0 | 100% | 0/11 | 0 | 0% | 0/11 |
| | | (100%) | (0%) | | (0%) | (0%) | | (0%) |
| | 1.0 | 48/50 | 0/50 | 96% | 0/50 | 0/50 | 0% | 2/50 |
| | | (96%) | (0%) | | (0%) | (0%) | | (4%) |
| | 5.0 | 45/70 | 9/70 | 77% | 7/70 | 2/70 | 13% | 7/70 |
| | | (64%) | (13%) | | (10%) | (3%) | | (10%) |
| | 7.0 | 78/123 | 12/123 | 73% | 26/123 | 3/123 | 23% | 4/123 |
| | | (63%) | (10%) | | (21%) | (2%) | | (3%) |

¹ZFN mRNA was injected into 1-cell embryos heterozygous for the golden^{b1} allele at the indicated dose.
²Embryos had at least one clone of unpigmented cells in an otherwise dark eye. Representative examples are shown in FIG. 1.
³Embryos had slight to moderate developmental defects. Common syndromes were a bent body axis or slight head necrosis.
⁴Embryos had severe developmental defects that precluded scoring of eye pigmentation mosaicism.

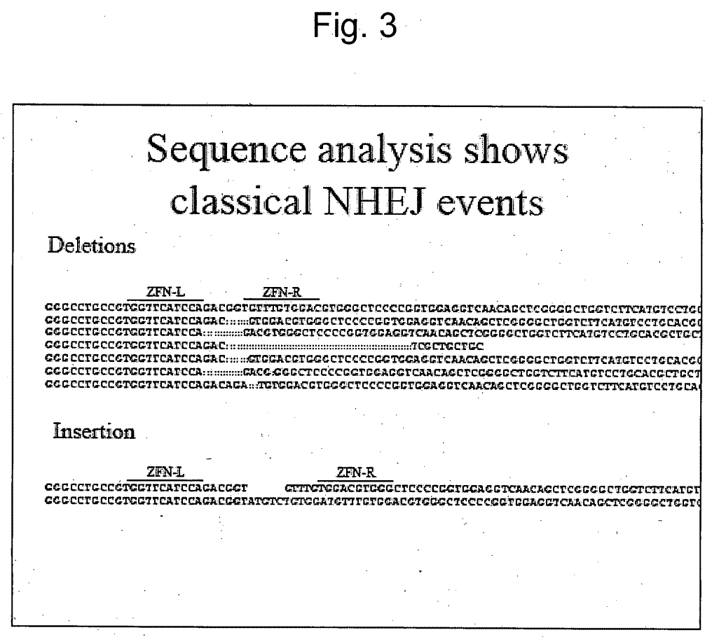

In addition, as shown in FIG. 3, sequence analysis as performed on various ZFN-treated embryos and showed the ZFN pairs induced both deletion and insertion mutations at the golden locus.

The codon in the golden locus at which the double-stranded break (DSB) was induced by the ZFN pairs was also determined and is indicated in Table A below by reference to the cognate amino acid number in the golden open reading frame (ORF).

TABLE A
Site of double-stranded break in
golden locus induced by ZFN pairs

| | Amino acid (numbered relative to ORF) at |
|---|---|
| | which DSB is induced in golden locus by |
| ZFN pair # | ZFN pairs |
| 1 | Ile 166 |
| 2 | Ile 166 |
| 3 | Ser 355 |
| 4 | Ser 355 |
| 5 | Asp 381 |
| 6 | Asp 381 |
| 7 | Asp 381 |
| 8 | Asp 381 |
| 9 | Asp 397 |
| 10 | Asp 397 |
| 11 | Val 399 |
| 12 | Ala 400 |
| 13 | Ala 400 |
| 14 | Val 437 |
| 15 | Val 437 |
| 16 | His 471 |
| 17 | His 471 |
| 18 | Glu 500 |
| 19 | Glu 500 |
| 20 | Glu 500 |
| 21 | Glu 500 |

### Example 2

### ZFNs Induce Targeted Disruption at the No Tail Locus

ZFNs targeted to various distinct positions in the no tail/Brachyury (ntl) locus were designed and incorporated into plasmids essentially as described in Umov et al. (2005) Nature 435(7042):646-651. ZFN pairs were screened for activity in a yeast-based chromosomal system as described in U.S. Ser. No. 60/995,566, entitled “Rapid in vivo Identification of Biologically Active Nucleases.”

The recognition helices for representative no tail zinc finger designs are shown below in Table 4.

TABLE 4
no tail Zinc finger Designs

| | | | | |
|---|---|---|---|---|
| ZFN Name | F1 | F2 | F3 | F4 |

| | | | | |
|---|---|---|---|---|
| 13368 notail | RSDTLSQ | DRSARTR | RSDDLSK | DNSNRIK |
| | (SEQ ID | (SEQ ID | (SEQ ID | (SEQ ID |
| | NO:27) | NO:28) | NO:29) | NO:30) |
| 13369 notail | RSDTLSQ | DRSARTR | RSDSLSK | DNSNRIK |
| | (SEQ ID | (SEQ ID | (SEQ ID | (SEQ ID |
| | NO:27) | NO:28) | NO:31) | NO:30) |
| 13370 notail | RSDNLSR | DSSTRKK | RSDHLSA | HSNARKT |
| | (SEQ ID | (SEQ ID | (SEQ ID | (SEQ ID |
| | NO:32) | NO:33) | NO:34) | NO:35) |

Target sites of the no tail zinc finger designs are shown below in Table 5.

TABLE 5
Target Sites of no tail Zinc Fingers

| | |
|---|---|
| ZFN Name | Target Site (5′ to 3′) |

| | |
|---|---|
| 13368 notail | tgTACTCGGTCCTGctggattttgtggc |
| | (SEQ ID NO:36) |
| 13369 notail | tgTACTCGGTCCTGctggattttgtggc |
| | (SEQ ID NO:37) |
| 13370 notail | gcATTAGGGTCGAGaccggtgacactgg |
| | (SEQ ID NO:38) |

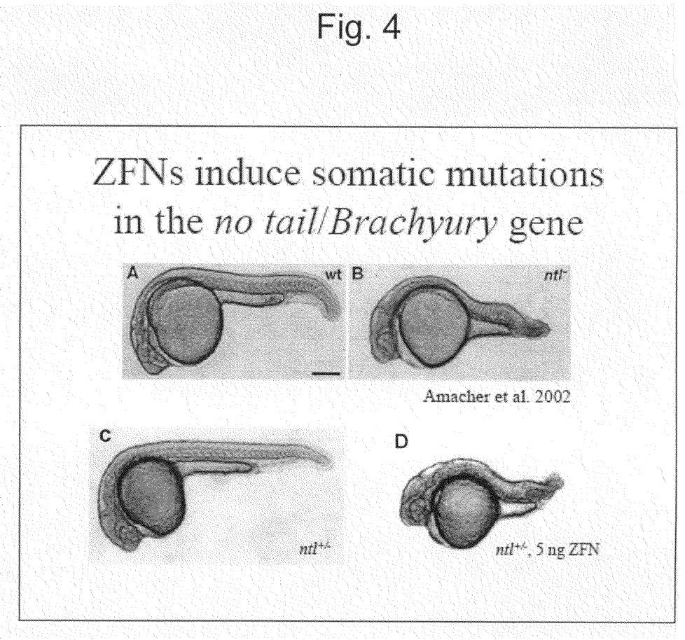

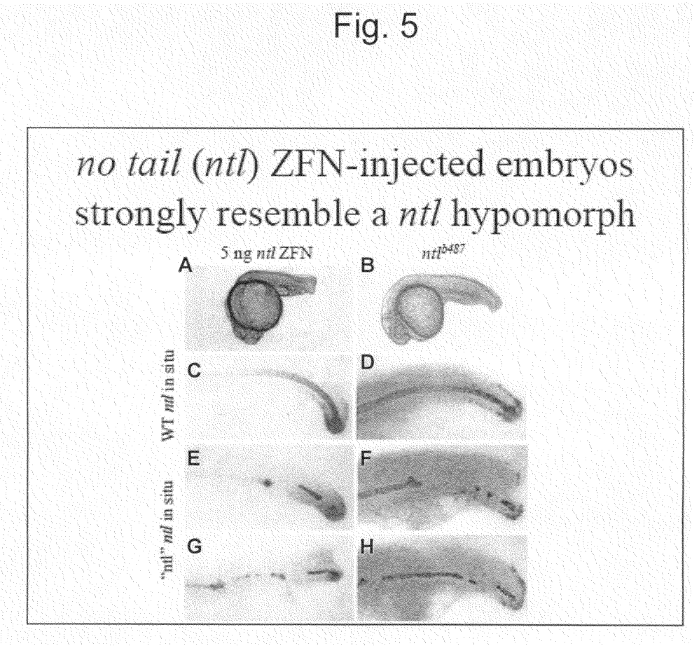

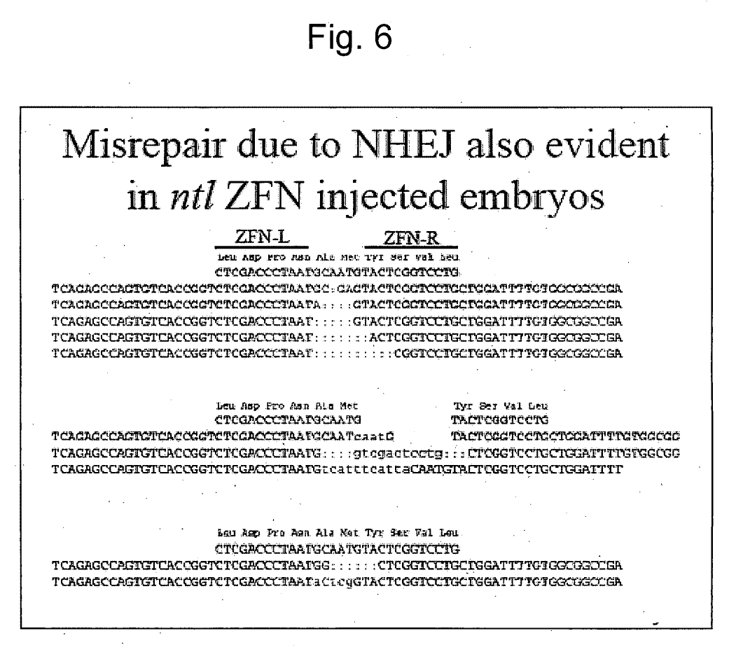

Active no tail-targeted ZFN mRNA was introduced by injection as described in Hammerschmidt et al. (1999) Methods Cell Biol. 59:87-115 into 1-cell zebrafish embryos from a cross between wildtype and ntl^{b195} heterozygous zebrafish. The injected embryos were then evaluated for ntl phenotype. 16-27% of injected embryos displayed a ntl-like phenotype (FIG. 4D), either mimicking the null phenotype (FIG. 4B) or a less severe phenotype typical of the hypomorphic allele, ntl^{b487} (Table 6, FIG. 5). Sequencing was performed on the region around the DSB site in the ZFN-injected embryos, and a broad range of deletions and insertions at the targeted locus was observed (FIG. 6).

TABLE 6
ZFNs directed to the zebrafish no tail locus
induce highly penentrant somatic mutation

| | | Wildtype | no tail-like | |
|---|---|---|---|---|
| ZFN pair¹ | Dose (ng) | embryos² | embryos³ | Unscored⁴ |
| ntl_78 | 5.0 | 68/91 (75%) | 15/91 (16%)⁵ | 8/91 (9%) |
| ntl_68 | 5.0 | 46/66 (70%) | 18/66 (27%)⁶ | 2/66 (3%) |

¹ZFN mRNA was injected into 1-cell embryos derived from a cross between ntl^{b195} heterozygote and wildtype individuals; approximately half the injected embryos were heterozygous for the ntl^{b195} allele and half did not carry the ntl^{b195} allele.
²At low magnification, embryos appeared wildtype by morphology. Some embryos were processed by in situ hybridization and more detect more subtle defects, such as gaps in the notochord, were observed (FIGS. 4 and 5).
³Embryos had a classic null or hypomorphic ntl phenotype (FIGS. 4 and 5)
⁴Embryos were too defective to score
⁵Of 15 embryos, 2 were slightly more necrotic than the typical ntl mutant
⁵Of 18 embryos, 5 were slightly more necrotic than the typical ntl mutant

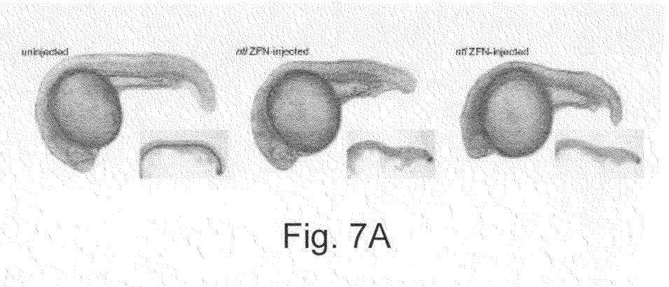

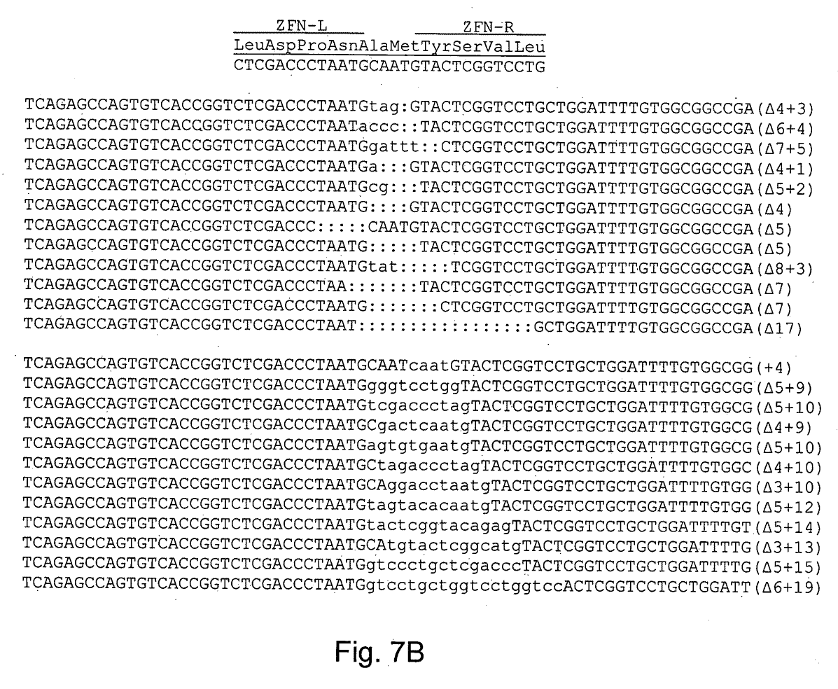

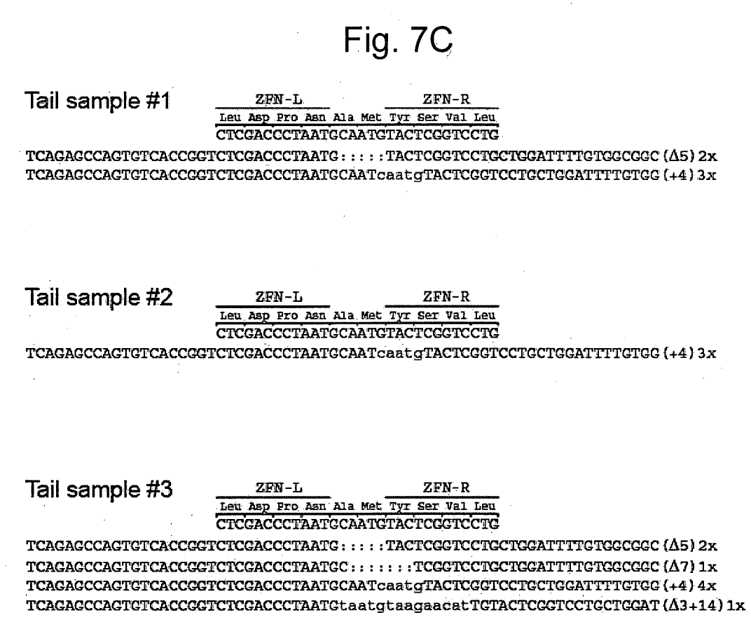

In addition, mRNA encoding no tail-targeting ZFNs were injected into wild-type embryos as described above. As shown in FIG. 7A, injection of ntl-targeted ZFNs in to wild-type embryos resulted in embryos exhibiting a ntl phenotype. Table 7 shows results of sequencing a 300 bp region surrounding the DSB site and shows that each of 2 representative embryos carried between 60-70% disrupted ntl alleles, respectively (see, also FIG. 7B).

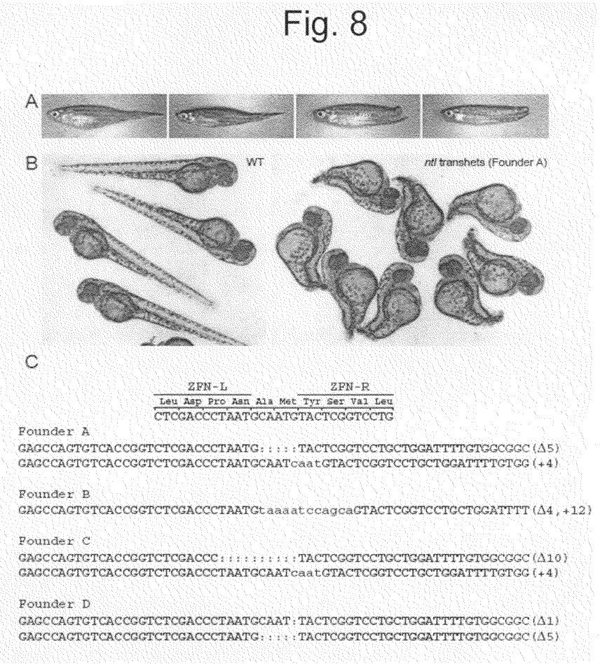

In addition, as shown in FIG. 7C, sequence analysis of DNA prepared from small posterior tissue samples obtained from adult tailless fish like those shown in FIG. 8A showed small deletions and insertions. The ntl locus surrounding the ZFN-cleavage site was amplified and sequenced. In every case, ntl mutant-bearing amplicons represent a significant fraction of the total (Sample 1, 5/25 (20%) ntl-bearing chromatids, 2 different alleles; Sample 2, 3/30 (10%) ntl-bearing chromatids, 1 allele; Sample 3, 8/29 (28%) ntl-bearing chromatids, 4 different alleles).

The codon in the ntl locus at which the double-stranded break (DSB) was induced by the ZFN pairs was also determined and is indicated in Table B below by reference to the cognate amino acid number in the ntl open reading frame (ORF).

TABLE B
Site of double-stranded break in ntl locus induced by ZFN pairs

| | Amino acid (numbered relative to ORF) at |
|---|---|
| ZFN pair # | which DSB is induced in ntl locus by ZFN pairs |
| 1 | Leu 14 |
| 2 | Ala 79 |
| 3 | Ala 79 |
| 4 | Trp 95 |
| 5 | Asn 124 |

Furthermore, when phenotypically wildtype embryos from these injections were raised, some juvenile and adult fish had posterior tail truncations (FIG. 8A). Given that a single wildtype ntl allele is sufficient for a normal phenotype, these data demonstrate the ability of these ZFNs to induce a biallelic disruption of a target gene locus.

TABLE 7

| ntl | | | Live | Scoreable | | | | |
|---|---|---|---|---|---|---|---|---|
| ZFN | Dose | Embryos | embryos at | embryos at | WT | ntl | WT ISH | ntl ISH |
| pair | (ng) | injected | 24 h | 24 h | phenotype | phenotype | phenotype | phenotype |
| 2 | 0.2 | 100 | 94/100 | 88/100 | 68/88 | 20/88 | N.D. | N.D. |
| | each | | (94%) | (88%) | (77%) | (23%) | | |
| | 1.0 | 101 | 87/101 | 60/101 | 17/60 | 43/60 | N.D. | N.D. |
| | each | | (86%) | (59%) | (28%) | (72%) | | |
| | 6.0 | 120 | 39/120 | 24/120 | 4/24 | 20/24 | 3/17 | 14/17 |
| | each | | (33%) | (20%) | (17%) | (83%) | (17%) | (83%) |

These results show the rapid generation of zebrafish mutants using ZFNs targeted to cleave in the gene of interest in the zebrafish genome and demonstrate that illicit DNA repair through the error-prone process of NHEJ at the site of cleavage resulted in functionally deleterious mutations and that ZFNs directed to the zebrafish no tail gene induce loss-of-function mutations on both chromatids early in development.

### Example 3

### ZFNs Induce Mutations in the Germline at the ntl Allele

To demonstrate that ZFNs can effectively induce mutations in the germline, wildtype embryos injected with no tail-targeting high-fidelity, obligate heterodimer ZFNs were raised to sexual maturity and screened. Eggs from ZFN-injected females were fertilized in vitro with sperm from males heterozygous for the ntl^{b195} allele.

Seven females analyzed and of these 4 generated ntl progeny (Table 8; FIG. 8B) at frequencies ranging between 1-13% as gauged by this complementation cross (Table 8). To measure the frequency of gametes carrying gene disruptions, the chromatid provided to the progeny (both wild-type and ntl) by four of the founder mothers was genotyped. The germline carried mutations at frequencies ranging from 5-28% (Table 8). Direct sequencing confirmed these estimates and revealed that three founders carried at least two new alleles, and one founder carried at least one (FIG. 8C).

TABLE 8

| Founder | Complementation testing data | | | | | Chromatid genotyping data** | | |
|---|---|---|---|---|---|---|---|---|
| ♀ | wt | ntl | unscored | % ntl progeny | % germline | wt progeny | ntl progeny | total |
| A | 109/118 | 9/118 | 0/118 | 7.6% | 15.3% | 10/96 | 9/9 | 19/105 |
| | 92.4% | 7.6% | | | | | | 18% |
| B | 78/79 | 1/79 | 0/79 | 1.3% | 2.5% | 1/38 | 1/1 | 2/39 |
| | 98.7% | 1.2% | | | | | | 5.1% |
| C | 37/50 | 3/50 | 10/50* | 6.0% | 12% | 8/37 | 3/3 | 11/40 |
| | 74% | 6% | 20% | | | | | 27.5% |
| D | 12/15 | 2/15 | 1/15* | 13.3% | 27% | 2/12 | 2/2 | 4/14 |
| | 80% | 13.3% | 6.4% | | | | | 28.5% |

*These progeny could not be conclusively phenotyped as being ntl and were excluded from the analysis.
**The ZFN target site overlaps a BsrDI restriction site. The chromatids were genotyped by amplifying the ZFN targeted stretch by PCR using primers that do not recognize the ntl^{b195}, and measuring the frequency of disrupted alleles by determining the fraction of BsrDI-resistant PCR products.

These results confirm that ZFNs can be used effectively to create heritable mutant alleles in loci of interest.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method of cleaving one or more paralogous or orthologous gene sequences in a zebrafish cell, the method comprising:
introducing, into the zebrafish cell, one or more zinc finger nucleases that bind to a target site in the zebrafish genome under conditions such that the zinc finger nucleases cleave the one or more paralogous or orthologous gene sequences.

2. The method of claim 1, wherein first and second zinc finger nucleases are used to cleave the gene sequences.

3. The method of claim 1, wherein the cleavage domain comprises a domain from a TypeIIS endonuclease.

4. The method of claim 3, wherein the TypeIIS endonuclease is FokI.

5. The method of claim 1, wherein the one or more zinc finger nucleases cleave in a coding region of the zebrafish genome.

6. The method of claim 1, wherein the one or more zinc finger nucleases cleave in a non-coding region of the zebrafish genome.

7. The method of claim 1, wherein the one or more zinc finger nucleases are introduced into the zebrafish cells as one or more polynucleotides encoding the one or more zinc finger nucleases.

8. The method of claim 7, wherein the polynucleotides are RNA.

9. A method for introducing an exogenous sequence into the genome of a zebrafish cell, the method comprising the steps of:
cleaving one or more paralogous genes of the genome of the zebrafish cell according to the method of claim 1; and
contacting the cell with an exogenous polynucleotide; such that cleavage of the paralogous genes stimulates integration of the exogenous sequence into the zebrafish genome by homologous recombination.

10. A method for modifying one or more gene sequences in the genome of a zebrafish cell, the method comprising
providing a zebrafish cell comprising one or more gene sequences; and
cleaving the genome of the zebrafish cell according to the method of claim 2,
wherein the first zinc finger nuclease cleaves at a first cleavage site and the second first zinc finger nuclease cleaves at a second cleavage site, wherein the gene sequence is located between the first cleavage site and the second cleavage site, and further wherein cleavage of the first and second cleavage sites results in modification of the gene sequences by non-homologous end joining.

11. The method of claim 10, wherein the non-homologous end joining results in a deletion between the first and second cleavage sites.

12. The method of claim 11, wherein the non-homologous end joining results in an insertion between the first and second cleavage sites.

13. A method of generating a zebrafish juvenile or adult carrying novel allelic forms of one or more selected genes, the method comprising:
modifying a cell of a zebrafish embryo according to the method of claim 10;
allowing the zebrafish embryo to develop into a juvenile or adult; and
selecting zebrafish juveniles or adults that carry novel allelic forms of the selected genes.

14. A method for germline disruption of one or more target genes in a zebrafish, the method comprising
modifying one or more gene sequences in the genome of one or more cells of a zebrafish embryo according to the method of claim 2; and
allowing the zebrafish embryo to reach sexual maturity,
wherein the modified gene sequences are present in at least a portion of gametes of the sexually mature zebrafish.

15. A method of creating one or more heritable mutant alleles in one or more zebrafish loci of interest, the method comprising
modifying one or more loci in the genome of one or more cells of a zebrafish embryo according to the method of claim 10;
raising the zebrafish embryo to sexual maturity; and
allowing the sexually mature zebrafish to produce offspring;
wherein some of the offspring comprise the mutant alleles.

16. A zinc finger nuclease comprising
a zinc finger protein comprising at least four zinc finger domains, wherein the zinc finger proteins comprise the recognition helices set forth in the rows of Table 1 and Table 4; and
a cleavage domain.

17. A polynucleotide encoding a zinc finger nuclease according to claim 16.

18. A zebrafish cell comprising one or more zinc finger nucleases according to claim 16.

19. A zebrafish cell comprising one or more polynucleotides according to claim 17.


SEQUENCE LISTING (92 sequences)

SEQ ID NO: 1 - protein, 5 aa, Artificial Sequence
  Synthetic peptide
TGEKP

SEQ ID NO: 2 - protein, 6 aa, Artificial Sequence
  Synthetic peptide
TGGQRP

SEQ ID NO: 3 - protein, 5 aa, Artificial Sequence
  Synthetic peptide
TGQKP

SEQ ID NO: 4 - protein, 6 aa, Artificial Sequence
  Synthetic peptide
TGSQKP

SEQ ID NO: 5 - protein, 7 aa, Artificial Sequence
  Synthetic peptide
DRSDLSR

SEQ ID NO: 6 - protein, 7 aa, Artificial Sequence
  Synthetic peptide
RSDDLTR

SEQ ID NO: 7 - protein, 7 aa, Artificial Sequence
  Synthetic peptide
QSGDLTR

SEQ ID NO: 8 - protein, 7 aa, Artificial Sequence
  Synthetic peptide
TSGSLSR

SEQ ID NO: 9 - protein, 7 aa, Artificial Sequence
  Synthetic peptide
RSDNLRE

SEQ ID NO: 10 - protein, 7 aa, Artificial Sequence
  Synthetic peptide
RSDALSE

SEQ ID NO: 11 - protein, 7 aa, Artificial Sequence
  Synthetic peptide
QNATRTK

SEQ ID NO: 12 - protein, 7 aa, Artificial Sequence
  Synthetic peptide
DRSHLSR

SEQ ID NO: 13 - protein, 7 aa, Artificial Sequence
  Synthetic peptide
RSDALAR

SEQ ID NO: 14 - protein, 7 aa, Artificial Sequence
  Synthetic peptide
DRSNLSR

SEQ ID NO: 15 - protein, 7 aa, Artificial Sequence
  Synthetic peptide
TSGSLTR

SEQ ID NO: 16 - protein, 7 aa, Artificial Sequence
  Synthetic peptide
QSGNLAR

SEQ ID NO: 17 - protein, 7 aa, Artificial Sequence
  Synthetic peptide
TSANLSR

SEQ ID NO: 18 - protein, 7 aa, Artificial Sequence
  Synthetic peptide
RSDTLSE

SEQ ID NO: 19 - protein, 7 aa, Artificial Sequence
  Synthetic peptide
RSQTRKT

SEQ ID NO: 20 - protein, 7 aa, Artificial Sequence
  Synthetic peptide
QSGNLAR

SEQ ID NO: 21 - protein, 7 aa, Artificial Sequence
  Synthetic peptide
TSGNLTR

SEQ ID NO: 22 - DNA, 28 bp, Artificial Sequence
  Synthetic oligonucleotide
gtgcagcgtgcggcctgctgtcctctgc

SEQ ID NO: 23 - DNA, 28 bp, Artificial Sequence
  Synthetic oligonucleotide
atgcacagcaggttatagacagcagaac

SEQ ID NO: 24 - DNA, 28 bp, Artificial Sequence
  Synthetic oligonucleotide
ttgttgacgtgggctccccggtggatgt

SEQ ID NO: 25 - DNA, 28 bp, Artificial Sequence
  Synthetic oligonucleotide
accgtctggatgaaccacggcaggccca

SEQ ID NO: 26 - DNA, 28 bp, Artificial Sequence
  Synthetic oligonucleotide
accgtctggatgaaccacggcaggccca

SEQ ID NO: 27 - protein, 7 aa, Artificial Sequence
  Synthetic peptide
RSDTLSQ

SEQ ID NO: 28 - protein, 7 aa, Artificial Sequence
  Synthetic peptide
DRSARTR

SEQ ID NO: 29 - protein, 7 aa, Artificial Sequence
  Synthetic peptide
RSDDLSK

SEQ ID NO: 30 - protein, 7 aa, Artificial Sequence
  Synthetic peptide
DNSNRIK

SEQ ID NO: 31 - protein, 7 aa, Artificial Sequence
  Synthetic peptide
RSDSLSK

SEQ ID NO: 32 - protein, 7 aa, Artificial Sequence
  Synthetic peptide
RSDNLSR

SEQ ID NO: 33 - protein, 7 aa, Artificial Sequence
  Synthetic peptide
DSSTRKK

SEQ ID NO: 34 - protein, 7 aa, Artificial Sequence
  Synthetic peptide
RSDHLSA

SEQ ID NO: 35 - protein, 7 aa, Artificial Sequence
  Synthetic peptide
HSNARKT

SEQ ID NO: 36 - DNA, 28 bp, Artificial Sequence
  Synthetic oligonucleotide
tgtactcggtcctgctggattttgtggc

SEQ ID NO: 37 - DNA, 28 bp, Artificial Sequence
  Synthetic oligonucleotide
tgtactcggtcctgctggattttgtggc

SEQ ID NO: 38 - DNA, 28 bp, Artificial Sequence
  Synthetic oligonucleotide
gcattagggtcgagaccggtgacactgg

SEQ ID NO: 39 - DNA, 87 bp, Artificial Sequence
  Synthetic oligonucleotide
gggcctgccgtggttcatccagacggtgtttgtggacgtgggctccccggtggaggtcaacagctcggggctggtcttcatgtcctg

SEQ ID NO: 40 - DNA, 84 bp, Artificial Sequence
  Synthetic oligonucleotide
gggcctgccgtggttcatccagacgtggacgtgggctccccggtggaggtcaacagctcggggctggtcttcatgtcctgcacg

SEQ ID NO: 41 - DNA, 82 bp, Artificial Sequence
  Synthetic oligonucleotide
gggcctgccgtggttcatccagacgtgggctccccggtggaggtcaacagctcggggctggtcttcatgtcctgcacgctgc

SEQ ID NO: 42 - DNA, 34 bp, Artificial Sequence
  Synthetic oligonucleotide
gggcctgccgtggttcatccagactcgctgctgc

SEQ ID NO: 43 - DNA, 85 bp, Artificial Sequence
  Synthetic oligonucleotide
gggcctgccgtggttcatccagacgtggacgtgggctccccggtggaggtcaacagctcggggctggtcttcatgtcctgcacgc

SEQ ID NO: 44 - DNA, 82 bp, Artificial Sequence
  Synthetic oligonucleotide
gggcctgccgtggttcatccagacggggctccccggtggaggtcaacagctcggggctggtcttcatgtcctgcacgctgct

SEQ ID NO: 45 - DNA, 86 bp, Artificial Sequence
  Synthetic oligonucleotide
gggcctgccgtggttcatccagacagatgtggacgtgggctccccggtggaggtcaacagctcggggctggtcttcatgtcctgca

SEQ ID NO: 46 - DNA, 83 bp, Artificial Sequence
  Synthetic oligonucleotide
gggcctgccgtggttcatccagacggtgtttgtggacgtgggctccccggtggaggtcaacagctcggggctggtcttcatgt

SEQ ID NO: 47 - DNA, 87 bp, Artificial Sequence
  Synthetic oligonucleotide
gggcctgccgtggttcatccagacggtatgtctgtggatgtttgtggacgtgggctccccggtggaggtcaacagctcggggctggt

SEQ ID NO: 48 - protein, 10 aa, Artificial Sequence
  Synthetic peptide
LDPNAMYSVL

SEQ ID NO: 49 - DNA, 30 bp, Artificial Sequence
  Synthetic oligonucleotide
ctcgaccctaatgcaatgtactcggtcctg

SEQ ID NO: 50 - DNA, 69 bp, Artificial Sequence
  Synthetic oligonucleotide
tcagagccagtgtcaccggtctcgaccctaatgcgagtactcggtcctgctggattttgtggcggccga

SEQ ID NO: 51 - DNA, 66 bp, Artificial Sequence
  Synthetic oligonucleotide
tcagagccagtgtcaccggtctcgaccctaatagtactcggtcctgctggattttgtggcggccga

SEQ ID NO: 52 - DNA, 65 bp, Artificial Sequence
  Synthetic oligonucleotide
tcagagccagtgtcaccggtctcgaccctaatgtactcggtcctgctggattttgtggcggccga

SEQ ID NO: 53 - DNA, 63 bp, Artificial Sequence
  Synthetic oligonucleotide
tcagagccagtgtcaccggtctcgaccctaatactcggtcctgctggattttgtggcggccga

SEQ ID NO: 54 - DNA, 60 bp, Artificial Sequence
  Synthetic oligonucleotide
tcagagccagtgtcaccggtctcgaccctaatcggtcctgctggattttgtggcggccga

SEQ ID NO: 55 - DNA, 70 bp, Artificial Sequence
  Synthetic oligonucleotide
tcagagccagtgtcaccggtctcgaccctaatgcaatcaatgtactcggtcctgctggattttgtggcgg

SEQ ID NO: 56 - DNA, 70 bp, Artificial Sequence
  Synthetic oligonucleotide
tcagagccagtgtcaccggtctcgaccctaatggtcgactcctgctcggtcctgctggattttgtggcgg

SEQ ID NO: 57 - DNA, 70 bp, Artificial Sequence
  Synthetic oligonucleotide
tcagagccagtgtcaccggtctcgaccctaatgtcatttcattacaatgtactcggtcctgctggatttt

SEQ ID NO: 58 - DNA, 64 bp, Artificial Sequence
  Synthetic oligonucleotide
tcagagccagtgtcaccggtctcgaccctaatggctcggtcctgctggattttgtggcggccga

SEQ ID NO: 59 - DNA, 70 bp, Artificial Sequence
  Synthetic oligonucleotide
tcagagccagtgtcaccggtctcgaccctaatactcggtactcggtcctgctggattttgtggcggccga

SEQ ID NO: 60 - DNA, 69 bp, Artificial Sequence
  Synthetic oligonucleotide
tcagagccagtgtcaccggtctcgaccctaatgtaggtactcggtcctgctggattttgtggcggccga

SEQ ID NO: 61 - DNA, 68 bp, Artificial Sequence
  Synthetic oligonucleotide
tcagagccagtgtcaccggtctcgaccctaataccctactcggtcctgctggattttgtggcggccga

SEQ ID NO: 62 - DNA, 68 bp, Artificial Sequence
  Synthetic oligonucleotide
tcagagccagtgtcaccggtctcgaccctaatggatttctcggtcctgctggattttgtggcggccga

SEQ ID NO: 63 - DNA, 67 bp, Artificial Sequence
  Synthetic oligonucleotide
tcagagccagtgtcaccggtctcgaccctaatgagtactcggtcctgctggattttgtggcggccga

SEQ ID NO: 64 - DNA, 67 bp, Artificial Sequence
  Synthetic oligonucleotide
tcagagccagtgtcaccggtctcgaccctaatgcgtactcggtcctgctggattttgtggcggccga

SEQ ID NO: 65 - DNA, 66 bp, Artificial Sequence
  Synthetic oligonucleotide
tcagagccagtgtcaccggtctcgaccctaatggtactcggtcctgctggattttgtggcggccga

SEQ ID NO: 66 - DNA, 65 bp, Artificial Sequence
  Synthetic oligonucleotide
tcagagccagtgtcaccggtctcgaccccaatgtactcggtcctgctggattttgtggcggccga

SEQ ID NO: 67 - DNA, 65 bp, Artificial Sequence; identical to SEQ ID NO: 52
  Synthetic oligonucleotide

SEQ ID NO: 68 - DNA, 65 bp, Artificial Sequence
  Synthetic oligonucleotide
tcagagccagtgtcaccggtctcgaccctaatgtattcggtcctgctggattttgtggcggccga

SEQ ID NO: 69 - DNA, 63 bp, Artificial Sequence; identical to SEQ ID NO: 53
  Synthetic oligonucleotide

SEQ ID NO: 70 - DNA, 63 bp, Artificial Sequence
  Synthetic oligonucleotide
tcagagccagtgtcaccggtctcgaccctaatgctcggtcctgctggattttgtggcggccga

SEQ ID NO: 71 - DNA, 53 bp, Artificial Sequence
  Synthetic oligonucleotide
tcagagccagtgtcaccggtctcgaccctaatgctggattttgtggcggccga

SEQ ID NO: 72 - DNA, 63 bp, Artificial Sequence
  Synthetic oligonucleotide
tcagagccagtgtcaccggtctcgaccctaatgcaatcaatgtactcggtcctgctggatttt

SEQ ID NO: 73 - DNA, 63 bp, Artificial Sequence
  Synthetic oligonucleotide
tcagagccagtgtcaccggtctcgaccctaatggggtcctggtactcggtcctgctggatttt

SEQ ID NO: 74 - DNA, 64 bp, Artificial Sequence
  Synthetic oligonucleotide
tcagagccagtgtcaccggtctcgaccctaatgtcgaccctagtactcggtcctgctggatttt

SEQ ID NO: 75 - DNA, 64 bp, Artificial Sequence
  Synthetic oligonucleotide
tcagagccagtgtcaccggtctcgaccctaatgcgactcaatgtactcggtcctgctggatttt

SEQ ID NO: 76 - DNA, 64 bp, Artificial Sequence
  Synthetic oligonucleotide
tcagagccagtgtcaccggtctcgaccctaatgagtgtgaatgtactcggtcctgctggatttt

SEQ ID NO: 77 - DNA, 65 bp, Artificial Sequence
  Synthetic oligonucleotide
tcagagccagtgtcaccggtctcgaccctaatgctagaccctagtactcggtcctgctggatttt

SEQ ID NO: 78 - DNA, 66 bp, Artificial Sequence
  Synthetic oligonucleotide
tcagagccagtgtcaccggtctcgaccctaatgcaggacctaatgtactcggtcctgctggatttt

SEQ ID NO: 79 - DNA, 66 bp, Artificial Sequence
  Synthetic oligonucleotide
tcagagccagtgtcaccggtctcgaccctaatgtagtacacaatgtactcggtcctgctggatttt

SEQ ID NO: 80 - DNA, 68 bp, Artificial Sequence
  Synthetic oligonucleotide
tcagagccagtgtcaccggtctcgaccctaatgtactcggtacagagtactcggtcctgctggatttt

SEQ ID NO: 81 - DNA, 69 bp, Artificial Sequence
  Synthetic oligonucleotide
tcagagccagtgtcaccggtctcgaccctaatgcatgtactcggcatgtactcggtcctgctggatttt

SEQ ID NO: 82 - DNA, 69 bp, Artificial Sequence
  Synthetic oligonucleotide
tcagagccagtgtcaccggtctcgaccctaatggtccctgctcgaccctactcggtcctgctggatttt

SEQ ID NO: 83 - DNA, 72 bp, Artificial Sequence
  Synthetic oligonucleotide
tcagagccagtgtcaccggtctcgaccctaatggtcctgctggtcctggtccactcggtcctgctggatttt

SEQ ID NO: 84 - DNA, 62 bp, Artificial Sequence
  Synthetic oligonucleotide
tcagagccagtgtcaccggtctcgaccctaatgtactcggtcctgctggattttgtggcggc

SEQ ID NO: 85 - DNA, 67 bp, Artificial Sequence
  Synthetic oligonucleotide
tcagagccagtgtcaccggtctcgaccctaatgcaatcaatgtactcggtcctgctggattttgtgg

SEQ ID NO: 86 - DNA, 60 bp, Artificial Sequence
  Synthetic oligonucleotide
tcagagccagtgtcaccggtctcgaccctaatgctcggtcctgctggattttgtggcggc

SEQ ID NO: 87 - DNA, 67 bp, Artificial Sequence
  Synthetic oligonucleotide
tcagagccagtgtcaccggtctcgaccctaatgtaatgtaagaacattgtactcggtcctgctggat

SEQ ID NO: 88 - DNA, 59 bp, Artificial Sequence
  Synthetic oligonucleotide
gagccagtgtcaccggtctcgaccctaatgtactcggtcctgctggattttgtggcggc

SEQ ID NO: 89 - DNA, 64 bp, Artificial Sequence
  Synthetic oligonucleotide
gagccagtgtcaccggtctcgaccctaatgcaatcaatgtactcggtcctgctggattttgtgg

SEQ ID NO: 90 - DNA, 64 bp, Artificial Sequence
  Synthetic oligonucleotide
gagccagtgtcaccggtctcgaccctaatgtaaaatccagcagtactcggtcctgctggatttt

SEQ ID NO: 91 - DNA, 54 bp, Artificial Sequence
  Synthetic oligonucleotide
gagccagtgtcaccggtctcgaccctactcggtcctgctggattttgtggcggc

SEQ ID NO: 92 - DNA, 63 bp, Artificial Sequence
  Synthetic oligonucleotide
gagccagtgtcaccggtctcgaccctaatgcaattactcggtcctgctggattttgtggcggc